(19) United States
(12) Patent Application Publication
(10) Pub. No.: US 2025/0325829 A1
(43) Pub. Date: Oct. 23, 2025

(54) LIGHT TREATMENT ASSEMBLY FOR USE WITHIN A SUPPORT DEVICE

(71) Applicant: Earthlite, LLC, Vista, CA (US)

(72) Inventors: James Chenevey, Encinitas, CA (US); Richard Estrada, Oceanside, CA (US); Angel Angeles, Escondido, CA (US)

(21) Appl. No.: 19/085,738

(22) Filed: Mar. 20, 2025

Related U.S. Application Data
(60) Provisional application No. 63/637,311, filed on Apr. 22, 2024.

(51) Int. Cl. A61N 5/06

(52) U.S. Cl. CPC A61N 5/06; A61N 2005/0659; A61N 2005/0663

(57) ABSTRACT

A support device (212) for supporting at least a portion of a user (10) relative to a surface (18) includes a device body (214) and a light treatment assembly (220). The device body (214) is configured to support the at least a portion of the user (10) relative to the surface (18). The device body (214) includes a resilient support layer (232) having a recessed pocket (238) formed therein. The light treatment assembly (220), such as a red light treatment assembly, a blue light treatment assembly or a near-infrared light treatment assembly, is embedded within the recessed pocket (238) of the resilient support layer (232).

### RELATED APPLICATION

This Application claims priority on U.S. Provisional Patent Application Ser. No. 63/637,311 filed on Apr. 22, 2024, and entitled “COLORED LIGHT TREATMENT ASSEMBLY FOR USE WITHIN A TREATMENT DEVICE”. To the extent permitted, the contents of U.S. Provisional Patent Application Ser. No. 63/637,311 are incorporated in their entirety herein by reference.

### BACKGROUND

As the benefits of therapeutic treatments such as therapeutic massage and/or other related treatments are becoming more widely appreciated, more and more people are participating in such therapeutic treatments. A typical treatment device, such as a massage table or a massage chair, allows the patient to be in a comfortable resting position while receiving a therapeutic massage or other therapeutic treatment.

Additionally, light therapy treatments, such as visible light therapy treatments, or low-level laser (light) therapy, is a fast-growing technology that exposes your skin to a lamp, device, or laser with a light panel incorporated therein in order to treat various issues or conditions for a patient. In particular, light therapy treatments have been used to treat a multitude of conditions that require stimulation of healing, relief of pain and inflammation, and restoration of function.

For example, red light therapy, also referred to as photobiomodulation, is a fast-growing therapeutic technique that has been utilized with some success for purposes of reducing pain and inflammation, repairing and/or healing muscle tissue, promoting injury recovery and healing of wounds, treating various skin issues or conditions such as lines, wrinkles, redness, acne, scars and other signs of aging, and promoting brain health. In particular, a red light therapy device uses low-level wavelengths of red light, such as between approximately 620 nanometers and 750 nanometers, to treat the patient, incorporating very low levels of heat that does not hurt or burn the skin. Such red light energy has been shown to penetrate deep into the skin and muscle tissue, thereby increasing blood flow. It is believed that a part of the cells called mitochondria can soak up the red light energy in order to make more energy. This can help cells repair themselves and become heathier, thus spurring healing in skin and muscle tissue.

Further, blue light therapy, using low-level wavelengths of blue light, such as between approximately 450 nanometers and 500 nanometers, is another therapeutic technique that has been used recently with some success for treating certain conditions on or just under the skin, such as sun damage, non-melanoma skin cancer treatment/prevention and acne. Blue light therapy has also seen certain levels of success in helping to overcome depression, and promoting sleep and mood improvement.

Still further, near-infrared light therapy, such as using wavelengths of between approximately 800 nanometers and 900 nanometers, is an innovative and non-invasive treatment that has been used to treat skin issues, to reduce pain and inflammation, and for certain therapeutic applications in ophthalmology.

It is important to note that the effectiveness of red light therapy, blue light therapy, and/or near-infrared light therapy depends on various factors, including the intensity and duration of the treatment, the distance between the light source and the skin, and the individual's skin type and condition.

As so described, low-level light therapy treatments, such as red light therapy, blue light therapy and near-infrared light therapy, are established therapy treatments. In most such therapy treatments, the tools and light panels used tend to be stand-alone items that are draped over the body, hung from the back of doors, or placed on the floor to be used. As a result, the users of such therapy treatments are often put in uncomfortable positions in order to use the therapy treatment. In some recent implementations, tanning beds have been repurposed for light therapy use. Unfortunately, all of these applications have experienced certain drawbacks such as either placing the user in contact with a plastic surface, often hard plastic, causing discomfort from sweat on an uncomfortable surface, or placing the user too far from the light source, thereby making it less effective.

### SUMMARY

The present invention is directed toward a support device for supporting at least a portion of a user relative to a surface. In various embodiments, the support device includes a device body and a light treatment assembly. The device body is configured to support the at least a portion of the user relative to the surface. The device body includes a resilient support layer. The light treatment assembly is embedded within the resilient support layer of the device body.

In some embodiments, the light treatment assembly includes a light panel having at least one light source.

In certain embodiments, the light treatment assembly further includes an assembly body that is configured to substantially encapsulate the light panel.

In some embodiments, the light treatment assembly is a red light treatment assembly; and the light panel includes at least one red light source that generates red light having a wavelength of between approximately 620 nanometers and 750 nanometers.

In other embodiments, the light treatment assembly is a blue light treatment assembly; and the light panel includes at least one blue light source that generates blue light having a wavelength of between approximately 450 nanometers and 500 nanometers.

In still other embodiments, the light treatment assembly is a near-infrared light treatment assembly; and the light panel includes at least one near-infrared light source that generates near-infrared light having a wavelength of between approximately 800 nanometers and 900 nanometers.

In many embodiments, the resilient support layer includes at least one foam layer.

In several embodiments, the resilient support layer has a recessed pocket formed therein; and wherein the light treatment assembly is embedded within the recessed pocket of the resilient support layer.

In many embodiments, the device body further includes a body cover that substantially surrounds at least a portion of the resilient support layer, the body cover including a cover aperture that is substantially aligned with the recessed pocket.

In several embodiments, the support device further includes a sheet cover that is configured to substantially surround at least a portion of the resilient support layer and the body cover, the sheet cover including a sheet aperture that is substantially aligned with the cover aperture and the recessed pocket.

In some embodiments, the support device further includes an aperture insert that is positioned within the sheet aperture, the aperture insert being configured to be substantially transparent to light from the light treatment assembly.

In various embodiments, the device body further includes a support platform; and the resilient support layer is positioned on and is supported by the support platform.

In certain embodiments, the resilient support layer has a recessed pocket formed therein; and the light treatment assembly is embedded within the recessed pocket of the resilient support layer.

In some embodiments, the support device further includes a foam border that is positioned to substantially encircle at least a portion of the light treatment assembly as embedded within the recessed pocket.

In many embodiments, the device body further includes a body cover that substantially surrounds at least a portion of the support platform and the resilient support layer, the body cover including a cover aperture that is substantially aligned with the recessed pocket.

In several embodiments, the support device further includes a sheet cover that is configured to substantially surround at least a portion of the support platform, the resilient support layer and the body cover, the sheet cover including a sheet aperture that is substantially aligned with the cover aperture and the recessed pocket.

In certain embodiments, the support device further includes an aperture insert that is positioned within the sheet aperture, the aperture insert being configured to be substantially transparent to light from the light treatment assembly.

In some embodiments, the support device is a treatment device that is configured to support a body of the user relative to the surface.

In other embodiments, the support device is a pillow assembly that is configured to support a head of the user relative to the surface.

The present invention is also directed toward a support device for supporting at least a portion of a user relative to a surface, including a device body that is configured to support the at least a portion of the user relative to the surface, the device body including (i) a support platform, (ii) a resilient support layer that is positioned on and is supported by the support platform, the resilient support layer including at least one foam layer having a recessed pocket formed therein, (iii) a body cover that substantially surrounds at least a portion of the support platform and the resilient support layer, the body cover including a cover aperture that is substantially aligned with the recessed pocket, and (iv) a sheet cover that is configured to substantially surround at least a portion of the support platform, the resilient support layer and the body cover, the sheet cover including a sheet aperture that is substantially aligned with the cover aperture and the recessed pocket, and an aperture insert that is positioned within the sheet aperture; and a light treatment assembly that is embedded within the recessed pocket of the resilient support layer, the light treatment assembly including a light panel having at least one light source, and an assembly body that is configured to substantially encapsulate the light panel; wherein the aperture insert is configured to be substantially transparent to light from the light treatment assembly.

The present invention is further directed toward a method for supporting at least a portion of a user relative to a surface, including supporting the at least a portion of the user relative to the surface with a device body, the device body including a resilient support layer; and embedding a light treatment assembly within the resilient support layer of the device body.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view illustration of a user and an embodiment of a support device having features of the present invention, the support device including a device body, and a light treatment assembly that is embedded within the device body, the support device being provided in the form of a treatment device;

FIG. 2 is an exploded perspective view illustration of a portion of an embodiment of the support device illustrated in FIG. 1;

FIG. 3 is an exploded perspective view illustration of a portion of another embodiment of the support device illustrated in FIG. 1;

FIG. 4A is a perspective view illustration of another embodiment of a support device having features of the present invention, the support device being provided in the form of a pillow assembly; and

FIG. 4B is an exploded perspective view illustration of the support device illustrated in FIG. 4A, the support device again including a device body, and an embodiment of the light treatment assembly that is embedded within the device body.

While embodiments of the present invention are susceptible to various modifications and alternative forms, specifics thereof have been shown by way of example and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

Embodiments of the present invention are described herein in the context of a light treatment assembly, such as a visible light treatment assembly in certain embodiments, for use within a support device. In alternative implementations, the support device can be a treatment device, such as a massage table or a massage chair, that is configured to support the body of a user or patient; or the support device can be a head supporting device, such as a pillow assembly, that is configured to support the head of the user or patient. In some embodiments, when the support device is a head supporting device, such as a pillow assembly, the head supporting device can be selectively couplable to a treatment device, or the head supporting device can be a stand-alone support device. In still other implementations, the support device can be incorporated into other suitable types of furniture.

In various embodiments, the support device includes a device body, and the light treatment assembly that is incorporated and/or embedded within the device body. In certain, non-exclusive, alternative embodiments, the light treatment assembly can be a visible light treatment assembly that can include a red light treatment assembly and/or a blue light treatment assembly. In other embodiments, the light treatment assembly need not be a visible light treatment assembly, but an rather be a non-visible light treatment assembly, such as near-infrared light treatment assembly. With such design, the user is able to conveniently receive a light therapy treatment at the same time that the user is receiving a therapeutic treatment, such as a massage. It is appreciated that in different implementations, the support device with the light treatment assembly incorporated and/or embedded therewithin could be useful in many different locations, such as in a spa, or in any suitable medical, physical therapy and wellness furniture fields.

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings. The same or similar nomenclature and/or reference indicators will be used throughout the drawings and the following detailed description to refer to the same or like parts.

In the interest of clarity, not all of the routine features of the implementations described herein are shown and described. It is appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is appreciated that such a development effort might be complex and time- consuming, but would nevertheless be a routine undertaking of engineering for those of ordinary skill in the art having the benefit of this disclosure.

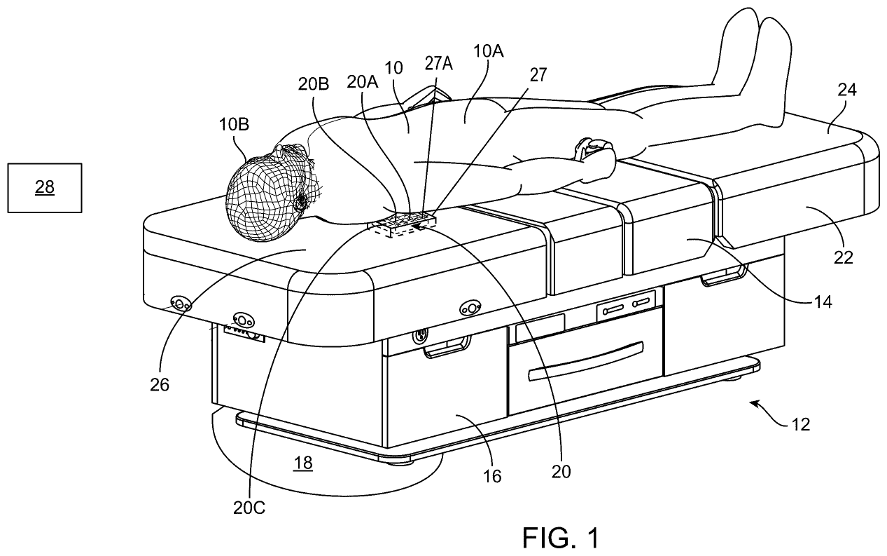

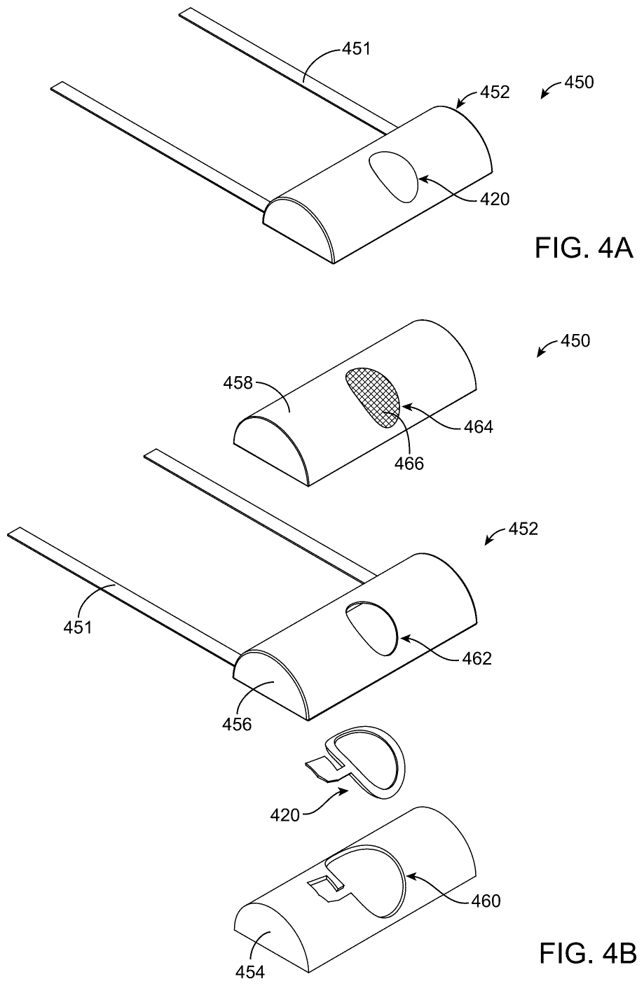

FIG. 1 is a perspective view illustration of a user 10 and an embodiment of a support device 12 having features of the present invention. As shown, the support device 12 is configured to support at least a portion of the user 10 relative to a surface 18, such as a floor or the ground. In some embodiments, such as illustrated in FIG. 1, the support device 12 can be provided in the form of a treatment device, such as a massage table or a massage chair, that supports a body 10A of the user 10 relative to the surface 18. In other embodiments, such as illustrated in FIG. 4A, the support device 12 can be provided in the form of a pillow assembly that supports a head 10B of the user 10 relative to the surface 18.

The design of the support device 12 can be varied to suit the needs of the user 10. As illustrated in the embodiment shown in FIG. 1, the support device 12 includes (i) a device body 14 that is configured to support the user 10 of the support device 12, (ii) a support assembly 16 that supports the device body 14 above and/or relative to the surface 18, (iii) a light treatment assembly 20 (illustrated in phantom) that is coupled to, incorporated within and/or embedded within the device body 14, and (iv) a control device 28 (illustrated as a box) that can be used to control various aspects of the support device 12, including control of the light treatment assembly 20. Alternatively, the support device 12 can include more components than those specifically illustrated in FIG. 1. For example, in certain non-exclusive alternative embodiments, the support device 12 can further include a headrest assembly (not shown) that is configured to support a face or head of the user 10 when the user 10 is positioned on the device body 14. Such headrest assembly can alternatively be coupled to the device body 14 or integrally formed into the device body 14. In one embodiment, the headrest assembly can include a support device 450 (illustrated in FIG. 4A) such as a pillow assembly that also includes features of the present invention.

As an overview, the support device 12 is configured to support the user 10 above the surface 18 as the user 10 is receiving a massage or other therapeutic treatment; and, at the same time, the light treatment assembly 20, which is coupled to, incorporated within and/or embedded within the device body 14 of the support device 12, is configured to provide desired light treatment therapy to the user 10 that is being supported on the support device 12. With such design, the support device 12 is directly usable both for therapeutic treatments such as therapeutic massage, and for light treatment therapy.

In some embodiments, the light treatment assembly 20 can be a red light treatment assembly that is configured to provide desired red light treatments to the user 10 as the user 10 is supported on the support device 12. In other embodiments, the light treatment assembly 20 can be a blue light treatment assembly that is configured to provide desired blue light treatments to the user 10 as the user 10 is supported on the support device 12. In still other embodiments, the light treatment assembly 20 can be a near-infrared light treatment assembly that is configured to provide desired near-infrared light treatments to the user 10 as the user 10 is supported on the support device 12. In yet other embodiments, the light treatment assembly 20 can be configured to provide other suitable or desired light treatments to the user 10 as the user 10 is supported on the support device 12.

As illustrated in FIG. 1, the support device 12 is shown as a treatment device in the form of a massage table that supports the user 10 relative to the surface 18, with the user 10 lying down while receiving a therapeutic treatment such as a massage. However, it is appreciated that the support device 12 can have another suitable design, and does not have to be provided in the form of a massage table. For example, in one non-exclusive alternative embodiment, the support device 12 can be provided as a treatment device in the form of a massage chair that supports the user 10 in a reclined and/or seated position relative to the surface 18 while receiving a therapeutic treatment such as a massage.

In the embodiment shown in FIG. 1, the device body 14 includes a plurality of body sections 22 that are movably coupled to one another such that an upper surface 24 of the support device 12 and/or the device body 14 can be adjusted to improve the comfort for the user 10 of the support device 12. In particular, in this embodiment, the device body 22 includes four body sections 22 that are movably coupled to one another so as to enable the adjustment of a height and/or an angle of the head, the torso and/or the legs of the user 10 relative to other body parts, regardless of the size of the user 10. Alternatively, the device body 14 can be formed with only a single body section.

As shown in this embodiment, the device body 14 is substantially rectangle-shaped. Alternatively, the device body 14 can have another suitable shape. For example, in certain non-exclusive alternative embodiments, the device body 14 can be substantially oval-shaped, oblong-shaped, rectangular-shaped with one or more rounded corners, or another suitable shape.

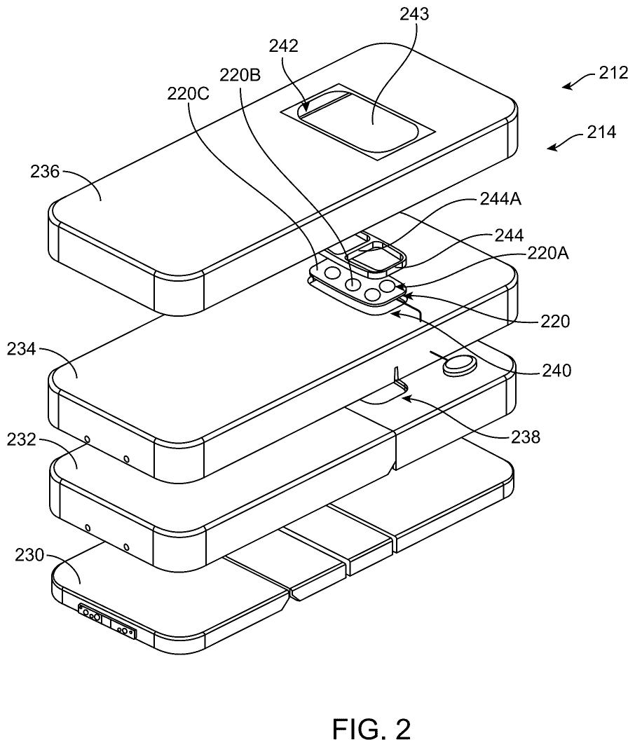

In many embodiments, the device body 14 includes a support platform 230 (illustrated in FIG. 2), a resilient support layer 232 (illustrated in FIG. 2), and a body cover 26. Alternatively, for example, the device body 14 can be made without the resilient support layer 232 and/or without the body cover 26.

The support platform 230 supports the user 10 receiving the treatment when the user 10 is positioned on the support device 12. The support platform 230 is generally rigid and can be made of a rigid material such wood, aluminum, steel, plastic or other suitable materials. In alternative, non-exclusive embodiments, the support platform 230 has a thickness of between approximately five millimeters and 30 millimeters. More particularly, the support platform 230 can have a thickness of approximately 5, 6, 7, 8, 9, 10, 11, 12, 13, 14, 15, 16, 17, 18, 19, 20, 21, 22, 23, 24, 25, 26, 27, 28, 29, or 30 millimeters. However, alternatively, the support platform 230 can have a thickness that is greater than or less than the noted thicknesses.

The resilient support layer 232 is secured to and can be positioned substantially on top of the support platform 230. The resilient support layer 232 provides a cushion for the comfort of the user 10 resting on the support device 12. Non-exclusive examples of suitable materials for the resilient support layer 232 include foam, memory foam, fleece pads, etc. The resilient support layer 232 can have any suitable thickness and any suitable stiffness/resiliency for purposes of comfortably supporting users 10 of many different sizes.

The body cover 26 secures the resilient support layer 232 to the support platform 230 and provides a protective covering for the resilient support layer 232. Non-exclusive examples of suitable materials for the body cover 26 include leather, plastic, vinyl, and cloth.

As described in greater detail herein below, in some embodiments, the body cover 26 can include a cover aperture 27 that coincides with the positioning of the light treatment assembly 20 within the device body 14. As further illustrated, in certain embodiments, an aperture insert 27A can be positioned or otherwise provided within the cover aperture 27. In one embodiment, the aperture insert 27A can be provided in the form of a mesh window that is configured to be substantially transparent to the light from one or more light sources 20B that can be included as part of the light treatment assembly 20. For example, the aperture insert 27A can be integrally formed into the body cover 26 or the aperture insert 27A can be secured to the body cover 26.

As further illustrated and described below in relation to subsequent Figures, in some embodiments, the device body 14 can further include a sheet cover 236 (illustrated in FIG. 2) that can be provided in the form of a fitted sheet that can be positioned to substantially surround at least the upper portion of the device body 14. However, it is appreciated that the device body 14 can be designed without the need for the sheet cover 236. No sheet cover 236 is shown in FIG. 1, at least in part, for purposes of clarity so that other components can be more easily seen and contemplated.

As noted above, the support assembly 16 supports the device body 14 above and/or relative to the surface 18. More particularly, as shown, the support assembly 16 extends between the device body 14 and the surface 18 to maintain the device body 14 positioned above and away from the surface 18. It is appreciated that the support assembly 16 can have any suitable design for purposes of supporting the device body 14 above and/or relative to the surface 18. For example, in one embodiment, the support assembly 16 can include one or more legs and/or feet that are fairly lightweight, but still provide desired stability for the support device 12. Alternatively, the support assembly 16 can have another suitable design.

In some embodiments, the support assembly 16 can be adjustable so as to enable a height of the device body 14 relative to the surface 18 to be selectively adjusted. For example, in one non-exclusive embodiment, the support assembly 16 can have legs that include individual leg members that are telescopingly coupled to one another to enable the selective height adjustment. Alternatively, the support assembly 16 can have another suitable design for purposes of enabling the desired selective height adjustment. It is appreciated that, in different embodiments, any such height adjustment can be accomplished manually or in an automated manner, such as through use of the control device 28.

In certain embodiments, the support assembly 16 can be further configured to provide one or more storage compartments, such as drawers, that are usable for storage of materials that can be used during any therapeutic treatments.

The light treatment assembly 20 is selectively activated to provide desired light treatments, such as (i) visible light treatments such as red light treatments, and/or blue light treatments; and/or (ii) near-infrared light treatments, to the user 10 as the user 10 is supported on the support device 12. In various embodiments, the light treatment assembly 20 is coupled to, incorporated within and/or embedded within the overall structure of the device body 14. Details of certain embodiments of how the light treatment assembly 20 is coupled to, incorporated within and/or embedded within the device body 14 will be described herein below in relation to subsequent Figures.

The design of the light treatment assembly 20 can be varied to suit the needs of the user 10 of the support device 12. In many embodiments, the light treatment assembly 20 can include a light panel 20A (illustrated in phantom), such as a visible light panel in certain non-exclusive embodiments, including the one or more light sources 20B (illustrated in phantom), such as one or more visible light sources in certain non-exclusive embodiments, and an assembly body 20C (illustrated in phantom) that is configured to substantially encapsulate the light panel 20A. Alternatively, the light treatment assembly 20 can have another suitable design.

Additionally, the size, shape and positioning of the light treatment assembly 20 can be varied depending on the specific light treatments that are to be administered to the user 10, and what part of the body of the user 10 is being treated. Moreover, it is appreciated that the support device 12 can include more than one light treatment assembly 20 depending on the needs and desires of the user 10 of the support device 12. For example, the light treatment assembly 20 can include multiple light panels 20A of multiple sizes, up to the full surface area of the device body 14.

In various embodiments, the light panel 20A can be an LED light therapy panel that can be embedded into one or more locations within the device body 14 of the support device 12. For example, in certain embodiments, a first light panel 20A can be embedded into the lower back area of the device body 14 and a second light panel 20A can be embedded into neck cushions to provide a more comfortable experience for the user 10. Alternatively, the light treatment assembly 20 can include any suitable number of light panels 20A which can be embedded in any other suitable locations within the device body 14. For example, an additional light panel (not shown) can be installed in the hamstring area or another altemative area. It should be noted that the multiple light panels can be controlled independently or in unison.

In some embodiments, each light source 20B can be a red light source that generates low-level wavelengths of red light, such as between approximately 620 nanometers and 750 nanometers. In other embodiments, each light source 20B can be a red light source that generates low-level wavelengths of red light, such as between approximately 650 nanometers and 700 nanometers. In still other embodiments, each light source 20B can be a red light source that generates low-level wavelengths of red light of approximately 660 nanometers. Alternatively, in yet other embodiments, each light source 20B can be a blue light source that generates low-level wavelengths of blue light, such as between approximately 450 nanometers and 500 nanometers. Still alternatively, in still yet other embodiments, each light source 20B can be a near-infrared light source that generates low-level wavelengths of near-infrared light, such as between approximately 800 nanometers and 900 nanometers.

The assembly body 20C is configured to provide a protective covering for the light panel 20A and/or for the individual light sources 20B encompassed within each light panel 20A. It is appreciated, however, that the assembly body 20C will be transparent to the light from the light panel 20A and/or the individual light sources 20B so as to not interfere with the light treatment therapy. In some embodiments, the assembly body 20C is provided in the form of a plastic cover that provides the desired transparent protective covering for the light panel 20A and/or for the individual light sources 20B encompassed within each light panel 20A. Alternatively, the assembly body 20B can have another suitable design.

As noted above, one common drawback for the typical use of light therapy is that it entails the user 10 having to lie directly on top of, or to otherwise directly contact, the assembly body 20C (the plastic cover for the light panel 20A). However, with the present invention, as described in greater detail herein below in relation to subsequent Figures, the support device 12 can further utilize a specially designed sheet cover 236 (illustrated in FIG. 2) so as to allow the user 10 to not to have to lay directly on, or otherwise directly contact, the assembly body 20C (the plastic cover) for the light panel 20A. It is appreciated that the use of such a specially designed sheet cover 236 helps to provide a breathable space between the user 10 and the light treatment assembly 20 while still maintaining an optimal distance for maximum benefit from the therapy.

It is further appreciated that the therapy provided by the light treatment assembly 20 can be controlled via wired or wireless handset, such as through use of the control device 28.

As noted, the control device 28 is configured to control various aspects of the support device 12. For example, in some embodiments, the control device 28 can be utilized to selectively adjust the support assembly 16 for purposes of selectively adjusting the height of the device body 14 relative to the surface 18.

In many embodiments, the control device 28 is further usable for purposes of controlling the light treatment assembly 20 so that the user 10 who is positioned on the support device 12 can receive the desired light treatment therapies in the desired areas of the body of the user 10. In FIG. 1, the control device 28 is illustrated as being positioned spaced apart from the support assembly 16. Alternatively, the control device 28 can be integrated into the support assembly 16.

FIG. 2 is an exploded perspective view illustration of a portion of the support device 212 illustrated in FIG. 1. In particular, FIG. 2 is an exploded perspective view illustration of an embodiment of the device body 214, which can include an embodiment of the light treatment assembly 220 coupled thereto and/or incorporated or embedded therewithin.

In certain embodiments, the light treatment assembly 220 can again be a red light treatment assembly that is configured to provide desired red light treatments to the user 10 (illustrated in FIG. 1) as the user 10 is supported on the support device 212. In other embodiments, the light treatment assembly 220 can again be a blue light treatment assembly that is configured to provide desired blue light treatments to the user 10 as the user 10 is supported on the support device 212. In still other embodiments, the light treatment assembly 220 can be configured to provide near- infrared light treatments to the user 10 as the user 10 is supported on the support device 212. In yet other embodiments, the light treatment assembly 220 can be configured to provide other suitable or desired light treatments to the user 10 as the user 10 is supported on the support device 212.

Additionally, as shown in FIG. 2, the light treatment assembly 220 can again include a light panel 220A, such as a visible light panel in certain non-exclusive embodiments, including one or more light sources 220B, such as one or more visible light sources in certain non-exclusive embodiments, and an assembly body 220C that is configured to substantially encapsulate the light panel 220A. The design and functionality of the light treatment assembly 220, and the components thereof, is substantially similar to what was illustrated and described herein above in relation to previous embodiments. Accordingly, a detailed description of the light treatment assembly 220, and the components thereof, will not be repeated in relation to FIG. 2.

Further, it is again appreciated that the support device 212 can include more than one light treatment assembly 220, and such light treatment assemblies 220 can be positioned within the device body 214 in any suitable manner in order to provide the desired light treatment therapy in the desired locations in order to effectively meet the needs and desires of the user 10 of the support device 212.

The design of the device body 214 can be varied to suit the requirements of the light treatment assembly 220 that is coupled thereto and/or incorporated or embedded therewithin. In alternative embodiments, the light treatment assembly 220 can include one or more light panels 220A that are mounted within the device body 214 in such a manner as to allow for greater comfort for the user 10 of the support device 212.

As illustrated, in certain embodiments, the device body 214 can include a support platform 230, a resilient support layer 232, a body cover 234, and a sheet cover 236. Alternatively, in other embodiments, the device body 214 can include more components or fewer components than what is specifically illustrated in FIG. 2.

The support platform 230 provides rigid support for the user 10 who is positioned on and supported by the support device 212. As shown, the support platform 230 is substantially rectangular-shaped. Alternatively, the support platform 230 can have another suitable shape. The support platform 230 is generally rigid and can be made of a rigid material such wood, aluminum, steel, plastic or other suitable materials. As noted above, in certain non-exclusive, alternative embodiments, the support platform 230 can have a thickness of between approximately five millimeters and 30 millimeters. More particularly, the support platform 230 can have a thickness of approximately 5, 6, 7, 8, 9, 10, 11, 12, 13, 14, 15, 16, 17, 18, 19, 20, 21, 22, 23, 24, 25, 26, 27, 28, 29, or 30 millimeters. However, alternatively, the support platform 230 can have a thickness that is greater than or less than the noted thicknesses.

The resilient support layer 232 can be secured to and can be positioned substantially on top of the support platform 230 to provide a cushion for the comfort of the user 10 resting on the support device 212. Non-exclusive examples of suitable materials for the resilient support layer 232 include foam, memory foam, fleece pads, etc. The resilient support layer 232 can have any suitable thickness and any suitable stiffness/resiliency for purposes of comfortably supporting users 10 of different sizes. In certain embodiments, the resilient support layer 232 can include a foam set comprised of one or more individual foam layers. In some embodiments, the resilient support layer 232 can include a combination of different foams. Alternatively, the resilient support layer 232 can have another suitable design.

As illustrated in FIG. 2, the resilient support layer 232 includes a recessed pocket 238 so that the light treatment assembly 220 can be positioned and/or embedded substantially therein. It is appreciated that the recessed pocket 238 can be positioned within the resilient support layer 232 in any suitable manner depending on the area(s) of the body of the user 10 to be treated using the light treatment assembly 220. Moreover, in some embodiments, the resilient support layer 232 can include a plurality of suitably positioned recessed pockets 238 to enable for the use of more than one light treatment assembly 220 if different areas of the body of the user 10 are to be treated at any given time.

As such, the present invention allows for mounting of light panels 220A of the light treatment assembly 220 into furniture, such as the support device 212, to allow for greater comfort. In this embodiment, the light panel 220A of the light treatment assembly 220 is mounted into the resilient support layer 232 within the recessed pocket 238 that is formed into the resilient support layer 232. This can be done in conjunction with the resilient support layer 232 including a combination of different foams, with the foams combining to allow for a balance of necessary support for the light panel 220A and softness for the body 10A (illustrated in FIG. 1) of the user 10. In certain implementations, the opening of the recessed pocket 238 can further wrap around the edges or perimeter of the light panel 220A to better hold the light panel 220A in place within the recessed pocket 238.

In many embodiments, the recessed pocket 238 is sized and shaped such that the light treatment assembly 220 fits relatively snugly therein, with a top surface of the assembly body 220C being substantially flush with an upper surface of the resilient support layer 232.

The body cover 234 can wrap around the resilient support layer 232 and secure the resilient support layer 232 to the support platform 230 in order to provide a supportive and protective covering for the resilient support layer 232. Non-exclusive examples of suitable materials for the body cover 234 include leather, plastic, vinyl, and cloth.

As shown in the embodiment illustrated in FIG. 2, the body cover 234 can include a cover aperture 240 that coincides with and/or is configured to be aligned with the recessed pocket 238 that is formed into the resilient support layer 232. With such design, the light therapy from the light panels 220A of the light treatment assembly 220 can be directed through the cover aperture 240.

The sheet cover 236 can be provided in the form of a fitted sheet that can be positioned to substantially surround at least the upper portion of the device body 214, including surrounding, at least in part, each of the support platform 230, the resilient support layer 232, and the body cover 234.

As shown, the sheet cover 236 can include an open, sheet aperture 242 that coincides with and/or is configured to be aligned with the cover aperture 240 and the recessed pocket 238. In certain embodiments, an aperture insert 243 can be positioned or otherwise provided within the sheet aperture 242. In such embodiments, the aperture insert 243 is configured to be substantially transparent to the light from the light sources 220B of the light treatment assembly 220. In some embodiments, the aperture insert 243 can be sewn or otherwise attached to the sheet cover 236 within the sheet aperture 242. In other embodiments, the aperture insert 243 can be integrally formed with the sheet cover 236 within the sheet aperture 242.

In one non-exclusive embodiment, the aperture insert 243 can be provided in the form of a mesh window that is transparent to the light and allows for the light therapy to come through the open, sheet aperture 242 and toward the body 10A of the user 10. As referred to herein, a “mesh window” is a window formed of a woven, knit, or knotted material of open texture with evenly spaced holes. Alternatively, the aperture insert 243 can have another suitable design. For example, suitable materials for the aperture insert 243 or mesh window can also include a perforated material for any suitable fabric or textiles, rubber, vinyl, polymer, or other suitable thin sheet materials.

As so described, the aperture insert 243, such as the mesh window, allows for a breathable space between the user 10 and the assembly body 220C and/or the light panel 220A of the light treatment assembly 220.

In certain non-exclusive alternative embodiments, it is appreciated that the device body 214 can be configured without the specific need and use of the sheet cover 236. In some such embodiments, the body cover 234 can instead incorporate a comparable aperture insert 27A (shown in FIG. 1), such as in the form of a mesh window, that can be positioned or otherwise provided within the cover aperture 240. In such embodiments, the aperture insert 27A would again be transparent to the light and allow for the light therapy to come through the cover aperture 240 and toward the body 10A of the user 10. However, it is further appreciated that the device body 214 will often include the sheet cover 236, as such design is easier for cleaning and comfort, as the sheet cover 236 can just be quickly and easily removed, cleaned and replaced as desired.

In yet another implementation, both the sheet cover 236 and the body cover 234 can be designed to include an aperture insert 243, 27A such as the mesh window.

In certain embodiments, as shown in FIG. 2, the support device 212 can further include a foam border 244 that is positioned about the light treatment assembly 220. The foam border 244 can help to maintain the positioning of the light treatment assembly 220 within the recessed pocket 238 while further enhancing the comfort of the user 10 of the support device 10. More specifically, in some embodiments, the foam border 244 can have one or more support members 244A to keep the user 10 from resting directly on the assembly body 220C and/or the light panel 220A of the light treatment assembly 220. In alternative embodiments, the foam border 244 can be a separate piece, or can be attached to the assembly body 220C and/or the light panel 220A via multiple methods.

As so illustrated and described, light therapy, such as (i) visible light therapy such as red light therapy, and/or blue light therapy; and/or (ii) near-infrared light therapy, has been incorporated and/or embedded into a recessed pocket 238 in the furniture to provide a more comfortable experience. In addition, the operator can use a special sheet cover 236 to allow people to not to have to lay directly on the assembly body 220C, which can be a plastic cover, of the light treatment assembly 220. As noted above, this is often a common complaint people have from the experience. Thus, as described, the incorporation and/or embedding of the light treatment assembly 220 into the device body 214 of the support device 212 provides a breathable space between the user 10 and the light source 220B, while still maintaining an optimal distance for maximum benefit from the light therapy.

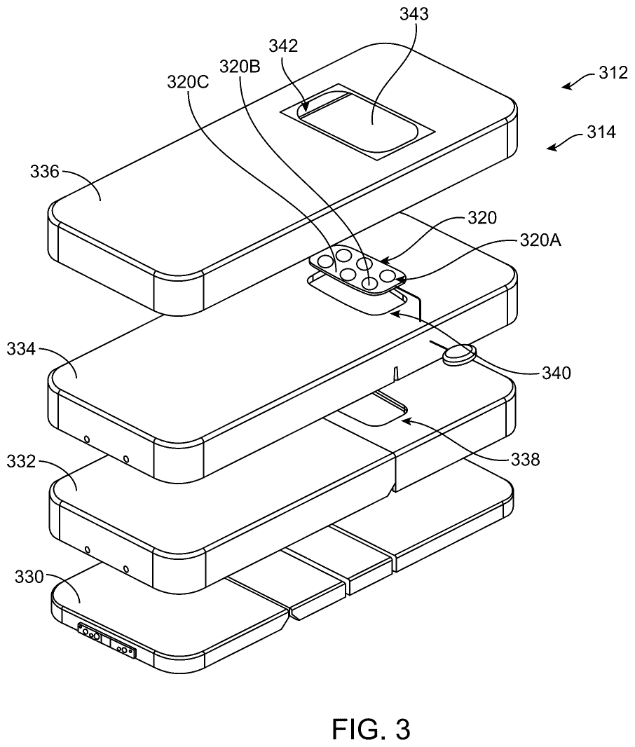

FIG. 3 is an exploded perspective view illustration of a portion of another embodiment of the support device 312 illustrated in FIG. 1. More particularly, FIG. 3 is an exploded perspective view illustration of another embodiment of the device body 314, which can again include an embodiment of the light treatment assembly 320 coupled thereto and/or incorporated or embedded therewithin.

In some embodiments, the light treatment assembly 320 can again be a light treatment assembly that is configured to provide desired light treatments (e.g., visible light treatments) to the user 10 (illustrated in FIG. 1) as the user 10 is supported on the support device 312. In a specific embodiments, the light treatment assembly 320 can again be a red and/or blue light treatment assembly that is configured to provide desired red and/or blue light treatments to the user 10 as the user 10 is supported on the support device 312. In still other embodiments, the light treatment assembly 320 can again be a near-infrared light treatment assembly that is configured to provide desired near-infrared light treatments to the user 10 as the user 10 is supported on the support device 312. In yet other embodiments, the light treatment assembly 320 can be configured to provide other suitable or desired light treatments to the user 10 as the user 10 is supported on the support device 312.

As shown in FIG. 3, the light treatment assembly 320 can again include a light panel 320A, such as a visible light panel in certain non-exclusive embodiments, including one or more light sources 320B, such as one or more visible light sources in certain non-exclusive embodiments, and an assembly body 320C that is configured to substantially encapsulate the light panel 320A. The design and functionality of the light treatment assembly 320, and the components thereof, is substantially similar to what was illustrated and described herein above in relation to previous embodiments. Accordingly, a detailed description of the light treatment assembly 320, and the components thereof, will not be repeated in relation to FIG. 3.

As with the previous embodiments, it is again appreciated that the support device 312 can include more than one light treatment assembly 320, and such light treatment assemblies 320 can be positioned within the device body 314 in any suitable manner in order to provide the desired light treatment therapy in the desired locations in order to effectively meet the needs and desires of the user 10 of the support device 312.

The embodiment of the device body 314 and the light treatment assembly 320 shown in FIG. 3 is substantially similar to the embodiment illustrated in FIG. 2. More specifically, the device body 314 once again includes a support platform 330, a resilient support layer 332 including a recessed pocket 338, a body cover 334 including a cover aperture 340, and a sheet cover 336 including a sheet aperture 342 and an aperture insert 343, which are substantially similar to the previous embodiment. Accordingly, a detailed description of such components will not be repeated in relation to FIG. 3. However, in this embodiment, the support device 312 and/or the device body 314 does not include the foam border that was included in the embodiment illustrated in FIG. 2.

FIG. 4A is a perspective view illustration of another embodiment of a support device 450 having features of the present invention. In particular, FIG. 4A is a perspective view illustration of the support device 450 that is being provided in the form of a pillow assembly, the support device 450 including a device body 452. FIG. 4A further illustrates an embodiment of the light treatment assembly 420 that can be incorporated and/or embedded within the device body 452.

In certain embodiments, the light treatment assembly 420 can again be a red light treatment assembly that is configured to provide desired red light treatments to the user 10 (illustrated in FIG. 1) as the head 10B (illustrated in FIG. 1) of the user 10 is supported on the support device 450. In other embodiments, the light treatment assembly 420 can again be a blue light treatment assembly that is configured to provide desired blue light treatments to the user 10 as the head 10B of the user 10 is supported on the support device 450. In still other embodiments, the light treatment assembly 420 can again be a near-infrared light treatment assembly that is configured to provide desired near-infrared light treatments to the user 10 as the user 10 is supported on the support device 412. In yet other embodiments, the light treatment assembly 420 can be configured to provide other suitable or desired light treatments to the user 10 as the head 10B of the user 10 is supported on the support device 450.

As shown in FIG. 4A, in certain embodiments, the support device 450 can have at least one attachment member 451 (two attachment members 451 are shown in FIG. 4A), such as at least one attachment strap, that is attached thereto. The attachment members 451 are configured to enable the support device 450, such as in the form of the pillow assembly, to be selectively attached to another support device, such as the support device 12 that is provided in the form of a treatment device as shown in FIG. 1. In certain embodiments, the attachment members 451 can be configured to wrap around at least a portion of the support device 12, such as at least a portion of the support platform 230 (illustrated in FIG. 2). In particular, in some embodiments, the attachment members can run to an underside of the support platform 230 where the attachment members can be secured to the support platform 230 in any suitable manner, such as through the use of hook-and-loop material. In such arrangement, the support device 450 can be configured to provide additional support to the head 10B of the user 10 relative to the surface 18, while the support device 12 is supporting the body 10A (illustrated in FIG. 1) of the user 10 relative to the surface 18.

Alternatively, in other implementations, the attachment members 451 can be utilized to selectively attach the support device 450 to another suitable device, such as another piece of furniture.

Still alternatively, the support device 450 can be designed as a stand-alone device in which there is no need to include the attachment members 451.

FIG. 4B is an exploded perspective view illustration of the support device 450 illustrated in FIG. 4A with an embodiment of the light treatment assembly 420 incorporated and/or embedded therewithin. FIG. 4B further illustrates the attachment members 451 that can be coupled to the support device 450 for purposes of selectively attaching the support device 450 to another support device and/or another piece of furniture.

The design of the support device 450, such as provided in the form of the pillow assembly, can be varied. In many embodiments, as shown, the support device 450 can include a device body 452 having a resilient support layer 454, a body cover 456, and a sheet cover 458. Alternatively, in other embodiments, the support device 450 and/or the device body 452 can include more components or fewer components than what is specifically illustrated in FIG. 4B. For example, in some embodiments, the support device 450 can further incorporate a support platform (not shown) for purposes of providing more rigid support for the head 10B (illustrated in FIG. 1) of the user 10 (illustrated in FIG. 1).

The resilient support layer 454 is configured to provide a cushion for the comfort and support of the head 10B of the user 10. Non-exclusive examples of suitable materials for the resilient support layer 454 include foam, memory foam, fleece pads, etc. The resilient support layer 454 can have any suitable size, shape and thickness. and any suitable stiffness/resiliency for purposes of comfortably supporting users 10 of different sizes. In certain embodiments, the resilient support layer 454 can include a foam matrix comprised of one or more individual foam layers of different sizes and/or shapes. In some embodiments, the resilient support layer 454 can include a combination of different foams. Alternatively, the resilient support layer 232 can have another suitable design.

As illustrated in FIG. 4B, the resilient support layer 454 includes a recessed pocket 460 so that the light treatment assembly 420 can be positioned and/or embedded therewithin. It is appreciated that the size, shape and overall design for the recessed pocket 460 can be varied to accommodate the particular size, shape and design of the light treatment assembly 420.

In this embodiment, the light treatment assembly 420 is mounted into the resilient support layer 454 within the recessed pocket 460 that is formed into the resilient support layer 454 in order to provide improved comfort for the user 10 while the user 10 is receiving the desired light therapy. This can be done in conjunction with the resilient support layer 454 including a combination of different foams, with the foams combining to allow for a balance of necessary support for the light treatment assembly 420 and softness for the head 10B of the user 10. The opening of the recessed pocket 460 can further wrap around the edges or perimeter of the light treatment assembly 420 to better hold the light treatment assembly 420 in place within the recessed pocket 460.

The body cover 456 can wrap around the resilient support layer 454 in order to provide a supportive and protective covering for the resilient support layer 454. Non- exclusive examples of suitable materials for the body cover 456 include leather, plastic, vinyl, and cloth.

As shown in the embodiment illustrated in FIG. 4B, the body cover 456 can include a cover aperture 462 that coincides with and/or is configured to be aligned with the recessed pocket 460 that is formed into the resilient support layer 454. With such design, the light therapy from the light treatment assembly 420 can be directed through the cover aperture 462.

The sheet cover 458 can be provided in the form of a fitted sheet that can be positioned to substantially surround at least the upper portion of the device body 452, including surrounding, at least in part, each of the resilient support layer 454, and the body cover 456.

As shown, the sheet cover 458 can include an open, sheet aperture 464 that coincides with and/or is configured to be aligned with the cover aperture 462 and the recessed pocket 460. In certain embodiments, an aperture insert 466 can be positioned or otherwise provided within the sheet aperture 464. In such embodiments, the aperture insert 466 is configured to be substantially transparent to the light from the light treatment assembly 420. In some embodiments, the aperture insert 466 can be sewn or otherwise attached to the sheet cover 458 within the sheet aperture 464. In other embodiments, the aperture insert 466 can be integrally formed with the sheet cover 458 within the sheet aperture 464.

In one non-exclusive embodiment, the aperture insert 466 can be provided in the form of a mesh window that is transparent to the light and allows for the light therapy to come through the open, sheet aperture 464 and toward the head 10B of the user 10. Alternatively, the aperture insert 466 can have another suitable design.

As so described, the aperture insert 466, such as the mesh window, allows for a breathable space between the user 10 and the light treatment assembly 420.

In some alternative embodiments, the support device 450 can be designed without the sheet cover 458, and a comparable aperture insert can instead be incorporated and/or positioned within the cover aperture 462 of the body cover 456.

It is appreciated that the light treatment assembly 420 will function in a similar manner as previously described, with the light panel of the light treatment assembly 420 being sized, shaped and positioned to coincide with and/or be aligned with the recessed pocket 460 of the resilient support layer 454, with the cover aperture 462 of the body cover 456, and with the sheet aperture 464 of the sheet cover 458. With such design, the light therapy can again be directed therethrough to provide the desired light therapy treatments, while still allowing for enhanced comfort for the user 10.

In summary, as described in detail herein, the present invention allows for embedding of light therapy panels into furniture and pillows to allow for greater comfort. In particular, by providing a support device having a device body with a light treatment assembly incorporated and/or embedded therewithin can provide various advantages for the user of the support device. Such advantages can include one or more of the following:

(1) The invention of building light therapy into a mattress topped table or chair or any other furniture or pillow, provides a more comfortable method for receiving light therapy. This allows a user to get the full benefit of the light therapy as they are not changing position of themselves or the light panel thereby reducing the therapy to the affected area; or cutting the therapy time short due to discomfort. It also keeps the light source close enough to the area being treated so as to be most effective.

(2) Embedding the light treatment assembly into the support device, such as a treatment device, eliminates the clutter of multiple devices in a small treatment room, while still allowing the user to receive multiple therapies, such as therapeutic massage and light therapy, substantially simultaneously.

(3) The upholstered body cover enables the treatment room and the support device to be used for other purposes aside from light therapy, thereby maximizing revenue of the treatment room and the support device.

It is understood that although a number of different embodiments of the support device have been illustrated and described herein, one or more features of any one embodiment can be combined with one or more features of one or more of the other embodiments, provided that such combination satisfies the intent of the present invention.

While a number of exemplary aspects and embodiments of the support device have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope, and no limitations are intended to the details of construction or design herein shown.

What is claimed is:

1. A support device for supporting at least a portion of a user relative to a surface, the support device comprising:
a device body that is configured to support the at least a portion of the user relative to the surface, the device body including a resilient support layer; and
a light treatment assembly that is embedded within the resilient support layer of the device body.

2. The support device of claim 1 wherein the light treatment assembly includes a light panel having at least one light source.

3. The support device of claim 2 wherein the light treatment assembly further includes an assembly body that is configured to substantially encapsulate the light panel.

4. The support device of claim 2 wherein the light treatment assembly is a red light treatment assembly; and wherein the light panel includes at least one red light source that generates red light having a wavelength of between approximately 620 nanometers and 750 nanometers.

5. The support device of claim 2 wherein the light treatment assembly is a blue light treatment assembly; and wherein the light panel includes at least one blue light source that generates blue light having a wavelength of between approximately 450 nanometers and 500 nanometers.

6. The support device of claim 2 wherein the light treatment assembly is a near-infrared light treatment assembly; and wherein the light panel includes at least one near-infrared light source that generates near-infrared light having a wavelength of between approximately 450 nanometers and 500 nanometers.

7. The support device of claim 1 wherein the resilient support layer includes at least one foam layer.

8. The support device of claim 7 wherein the resilient support layer has a recessed pocket formed therein; and wherein the light treatment assembly is embedded within the recessed pocket of the resilient support layer.

9. The support device of claim 8 wherein the device body further includes a body cover that substantially surrounds at least a portion of the resilient support layer, the body cover including a cover aperture that is substantially aligned with the recessed pocket.

10. The support device of claim 9 further comprising a sheet cover that is configured to substantially surround at least a portion of the resilient support layer and the body cover, the sheet cover including a sheet aperture that is substantially aligned with the cover aperture and the recessed pocket.

11. The support device of claim 10 further comprising an aperture insert that is positioned within the sheet aperture, the aperture insert being configured to be substantially transparent to light from the light treatment assembly.

12. The support device of claim 7 wherein the device body further includes a support platform; and wherein the resilient support layer is positioned on and is supported by the support platform.

13. The support device of claim 12 wherein the resilient support layer has a recessed pocket formed therein; and wherein the light treatment assembly is embedded within the recessed pocket of the resilient support layer.

14. The support device of claim 13 further comprising a foam border that is positioned to substantially encircle at least a portion of the light treatment assembly as embedded within the recessed pocket.

15. The support device of claim 13 wherein the device body further includes a body cover that substantially surrounds at least a portion of the support platform and the resilient support layer, the body cover including a cover aperture that is substantially aligned with the recessed pocket.

16. The support device of claim 15 further comprising a sheet cover that is configured to substantially surround at least a portion of the support platform, the resilient support layer and the body cover, the sheet cover including a sheet aperture that is substantially aligned with the cover aperture and the recessed pocket.

17. The support device of claim 16 further comprising an aperture insert that is positioned within the sheet aperture, the aperture insert being configured to be substantially transparent to light from the light treatment assembly.

18. The support device of claim 1 wherein the support device is a treatment device that is configured to support a body of the user relative to the surface.

19. The support device of claim 1 wherein the support device is a pillow assembly that is configured to support a head of the user relative to the surface.

20. A support device for supporting at least a portion of a user relative to a surface, the support device comprising:
a device body that is configured to support the at least a portion of the user relative to the surface, the device body including (i) a support platform, (ii) a resilient support layer that is positioned on and is supported by the support platform, the resilient support layer including at least one foam layer having a recessed pocket formed therein, (iii) a body cover that substantially surrounds at least a portion of the support platform and the resilient support layer, the body cover including a cover aperture that is substantially aligned with the recessed pocket, and (iv) a sheet cover that is configured to substantially surround at least a portion of the support platform, the resilient support layer and the body cover, the sheet cover including a sheet aperture that is substantially aligned with the cover aperture and the recessed pocket, and an aperture insert that is positioned within the sheet aperture; and
a light treatment assembly that is embedded within the recessed pocket of the resilient support layer, the light treatment assembly including a light panel having at least one light source, and an assembly body that is configured to substantially encapsulate the light panel;
wherein the aperture insert is configured to be substantially transparent to light from the light treatment assembly.
